(19) United States
(12) Patent Application Publication
(10) Pub. No.: US 2026/0174814 A1
(43) Pub. Date: Jun. 25, 2026

(54) COMPOSITION FOR TREATING OR ALLEVIATING MUSCLE LOSS AND MUSCLE ATROPHY CONTAINING OREGONIN COMPOUND DERIVED FROM ALNUS PLANT AS ACTIVE INGREDIENT

(71) Applicant: DR. OREGONIN INC., Chuncheon-si (KR)

(72) Inventor: Sun Eun CHOI, Chuncheor-si (KR)

(21) Appl. No.: 18/727,306

(22) PCT Filed: Apr. 11, 2023
(86) PCT No.: PCT/KR2023/004858

§ 371 Date: Jul. 8, 2024

(30) Foreign Application Priority Data
Jan. 30, 2023 (KR) 10-2023-0012180
Apr. 10, 2023 (KR) 10-2023-0046995

(51) Int. Cl. A61K 36/185; A23K 10/30; A23K 20/163; A61K 31/7034; A61P 21/00

(52) U.S. Cl. CPC A61K 36/185; A23K 10/30; A23K 20/163; A61K 31/7034; A61P 21/00; A61K 2236/333; A61K 2236/37; A61K 2236/39

(57) ABSTRACT

Provided is a pharmaceutical composition for preventing and treating muscle loss, comprising an oregonin compound derived from a plant of the genus Alnus as an active ingredient.

### TECHNICAL FIELD

The present invention relates to a composition for treating or alleviating muscle loss and muscle atrophy containing an oregonin compound derived from Alnus plant as an active ingredient.
Related task number: 2023469A00-2325-EE01
Department name: Korea Forest Service
Task management (specialized) agency name: The Korea Forestry Promotion Institute
Research project title: Technology development based on the utilization of forest biological materials/technology development for standardizing functional raw materials of forest resources
Research task title: Development of natural materials for new muscle loss using tree resources of Alnus and establishment of smart forest bio-production technology
Contribution ratio of 1/1
Name of the task execution organization: Kangwon National University Industry-Academic Cooperation Foundation
Research period: 2023-04-01 to 2025-12-31

### BACKGROUND ART

Muscles can be divided into skeletal muscle, smooth muscle, and cardiac muscle in terms of structure or function. Among them, skeletal muscle is about 600 voluntary muscles that are directly under the skin of the hands, feet, chest, abdomen, etc. and attached to the bones through the whole body's bones or tendons. It is suitable for moving or supporting bone through contraction. The contraction is caused and regulated by the neural signals. It accounts for 40-50% of your body weight and functions to maintain body temperature and generate energy. The micromyofibrils of actin and myosin are regularly arranged so that the horizontal pattern can be observed on the microscope (Lieber R. L., 2002; Edwards R. H., 1981).

Skeletal muscle fibers are divided into three biochemical categories: Type I, Type IIa, and Type IIb according to the content of mitochondria. A posture-maintaining root that is made of red slow fibers and maintains a weak force for a long time to maintain the posture is called Type I. It is a muscle suitable for exercise such as aerobic long-distance running due to its high mitochondrial content. Among the fast muscle fibers, those having the characteristics of slow muscle fibers are called Type IIa. When making movements, muscles made of white fast muscle fibers are used, which are called active muscles and are classified as Type IIb. The low content of mitochondria makes it a suitable muscle for exercise, such as anaerobic short-distance running. These skeletal muscle fibers are distributed in different proportions in each part of the body (Tortora et al, 2008).

Muscle atrophy is described by the anti-anabolic and catabolic action of unbalanced muscle fibers. Here, muscle atrophy refers to the size and mass loss of muscle cells and muscle tissue when the muscle is not used due to aging, disease conditions (excessive exposure to stress hormones, cancer, sepsis, starvation, etc.), and decreased activity of pathological life. When muscle atrophy occurs, muscle strength for physical activity is weakened, and the vicious cycle of musculoskeletal degeneration begins. Reduction in walking speed and deterioration of grip strength are the main symptoms and indicators of muscle mass reduction, and may lead to falls, fractures, joint damage, metabolic disorders, and cardiovascular diseases.

glucocorticoids in our body causes molecular biological changes in muscle fibers and is directly or indirectly involved in anti-assimilation and catabolism. dexamethasone, which is a Glucocorticoids-based compound, serves to inhibit PI3K/Akt/mTOR pathway as an anti-anabolic action, which inhibits the activities of 4E-BP1 and S6K1, which are downstream effectors, thereby preventing the operations of eIF4G (Eukaryotic translation initiation factor 4G) and eIF4E (Eukaryotic translation initiation factor 4E). This inhibits the mRNA translation process for protein synthesis, which is shown as muscle fiber atrophy caused by inhibition of muscle fiber synthesis and protein degradation (Shackman et al., 2013).

Dexamethasone also induces muscle atrophy by causing synthesis inhibition and proteolysis of muscles. This expresses the gene atrogene (Atrogin-1, MuRF-1) that induces muscle atrophy according to the mechanism leading to “PI3K/Akt→FOXO activation and GSK3 inactivation”, and these genes induce proteolysis represented by ubiquitin-proteasome system. Therefore, it is necessary to develop a material for preventing and treating muscle loss, a disease in which skeletal muscle is reduced.

### DISCLOSURE

### Technical Problem

Accordingly, the problem to be solved by the present invention is to provide a substance for treating or alleviating muscle loss and muscle atrophy.

### Technical Solution

To achieve the above object, the present invention provides a composition for treating or alleviating muscle loss and muscle atrophy, which comprises an oregonin compound derived from a plant of the genus Alnus as an active ingredient.

In an embodiment of the present invention, the oregonin compound derived from a plant of Alnus japonica is an extract of the supercritical extract meal of a plant of Alnus japonica.

In an embodiment of the present invention, an oregonin compound derived from a plant of Alnus japonica is included as an active ingredient.

In an embodiment of the present invention, the oregonin compound derived from a plant of Alnus japonica is an extract of the supercritical extraction meal of a plant of Alnus japonica, and the oregonin compound derived from a plant of Alnus japonica is obtained by solvent fractionating the supercritical extraction meal of Alnus japonica.

In an embodiment of the present invention, the plant of the genus Alnus is a root of a plant of the genus Alnus. muscle loss, comprising an oregonin compound derived from a plant of the genus Alnus as an active ingredient

The present invention also provides a food composition for preventing and alleviating muscle loss, comprising an oregonin compound derived from a plant of the genus Alnus as an active ingredient.

In an embodiment of the present invention, the oregonin compound derived from a plant of Alnus japonica is an extract of the supercritical extraction meal of a plant of Alnus japonica, and the oregonin compound derived from a plant of Alnus japonica is obtained by solvent fractionating the supercritical extraction meal of Alnus japonica.

In an embodiment of the present invention, the plant of the genus Alnus is a root of a plant of the genus Alnus.

The present invention also provides a feed composition for preventing and alleviating muscle loss, which includes an oregonin compound derived from a plant of the genus Alnus as an active ingredient.

In an embodiment of the present invention, the oregonin compound derived from a plant of Alnus japonica is an extract of the supercritical extraction meal of a plant of Alnus japonica, and the oregonin compound derived from a plant of Alnus japonica is obtained by solvent fractionating the supercritical extraction meal of Alnus japonica.

In an embodiment of the present invention, the plant of the genus Alnus is a root of a plant of the genus Alnus.

### Advantageous Effects

The pharmaceutical composition for preventing and treating muscle loss according to the present invention is based on an oregonin compound derived from a plant of the genus Alnus and has an effect of inhibiting muscle loss against hydrogen peroxide or dexamethasone.

### DESCRIPTION OF DRAWINGS

FIG. 1 is testing material information according to an embodiment of the present disclosure.

FIG. 2 shows cell viability (ORE) of C2C12 cells under normal conditions according to an embodiment of the present invention.

FIG. 3 shows cell viability (ORE) of C2C12 cells in H2O2 treatment conditions according to an embodiment of the present invention.

FIG. 4 is an apoptosis (ORE) of C2C12 cells under H2O2 treatment conditions according to an embodiment of the present invention.

FIG. 5 shows the cell viability (ORE) of C2C12 cells in Dexamethasone treatment conditions according to an embodiment of the present invention.

### BEST MODE

Hereinafter, specific embodiments of the present invention will be described with reference to the drawings. However, this is merely an example, and the present invention is not limited thereto.

In describing the present disclosure, when it is determined that a detailed description of a known technology related to the present disclosure may unnecessarily obscure the subject matter of the present disclosure, the detailed description thereof will be omitted. Further, terms to be described below are terms defined in consideration of functions in the present disclosure, and may vary according to the intention or custom of a user or an operator. Therefore, the definition should be made based on the contents throughout this specification.

The technical idea of the present disclosure is determined by Claims, and the following embodiments are merely means for efficiently explaining the technical idea of the present disclosure to those skilled in the art to which the present disclosure pertains.

The present invention provides a pharmaceutical composition for preventing and treating muscle loss, based on an inhibitory effect of an oregonin compound derived from a plant of the genus Oryzae that is described below on the decomposition of skeletal muscle and the death of myofibroblasts, and a food composition for preventing or alleviating muscle loss. The composition according to the present invention is also a composition for feed of companion animals, and may be utilized as an additive or the like.

Hereinafter, the present invention will be described in more detail through Examples and Experimental Examples.

### EXAMPLES

### 1. Materials and Methods

### 1.1. Material of Disclosure

In August 2021, 15.25 kg of Alnus sibirica Fisch. Ex Turcz. root was obtained from the test forest of the Forest Research Institute located in Chuncheon, Gangwon-do (921-2, Jinae-ri, Chuncheon, Gangwon-do). 14.25 kg of the root of the Alnus japonica was subjected to 60% ethanol extraction to obtain AS60E, and 1 kg of the root of the Alnus japonica was subjected to supercritical extraction to obtain 60% ethanol extraction from the supercritical extraction foil, a by-product, to obtain ASRFI. In addition, ASRFI, which is a solvent fraction of a supercritical extract meal 60% of a spirit extract, was subjected to ethyl acetate solvent fractionation to obtain ASRF-II, and a solvent fraction of a 60% of a root extract of a Alnus japonica was subjected to ethyl acetate solvent fractionation to obtain ASRF-III. Next, compound1, which is oregonin in the form of powder obtained through separation purification, was obtained and LC-MS/MS and NMR. analysis were conducted at the Joint Experimental Laboratory Center of Kangwon National University. Standard products are being stored at the Forest Bio Materials Engineering Department of the Forest Environment Science University of Kangwon National University and the Arboretum Natural Products Functional Materials Laboratory.
   1.2. Extract of Alnus japonica

14.25 kg of Alnus sibirica Fisch. Ex Turcz. root was immersed in 60% edible alcohol alcohol alcohol alcohol alcohol alcohol alcohol alcohol and 40% distilled water at 25° C. for 1 week to perform extraction three times, and then filtered with filter paper (Hyundai Micro, Seoul, Korea) having a size of 594 mm×941 mm. Water bath was concentrated using RE-501 (Lanphan, Henan, China) and low temperature coolant circulation pump was concentrated using DLSB-5L/−20° C. (Lanphan, Henan, China). Next, freeze dryer (Ilshin, Gyeonggi-do, Korea) was set to a temperature of −80° C., and then concentrated Alnus sibirica Fisch. Ex Turcz. 60% ethanol extract was freeze-dried for 48 hours to obtain a final 428.32 g (yield 3.01%, AS60E). In addition, for supercritical extraction, 1 kg of the root of Alnus sibirica Fisch. Ex Turcz. dried by using ISA-SEFE-0500-0700-080 (Ilsin, Daejeon, Korea), which is equipment for supercritical extraction research, was added thereto, and carbon dioxide gas was maintained at isostatic pressure, and then the temperature of the extraction tank was set to 50° C. Next, a pressure of 400 bar was applied using a high-pressure pump, and then a total of 180 mL of edible alcohol (ethanol) was added to the lower portion of the extraction tank at 3 mL/min for 1 hour (Seo et al., 2018; Ha et al 2019; Choi et al 2019). The extraction was carried out by adding carbon dioxide using a high-pressure pump for 30 minutes while maintaining the temperature and pressure, and then 970.2 g of a sample of supercritical extract cucurbitum of an oak tree, which is a residue, was dried, extracted with 60% alcohol at room temperature, filtered, and concentrated to obtain a final 17.27 g (1.78% ASRF-I).

### 1.3. Isolation of Extracts

200 g of 428.32 g of the powdered powder obtained through the Alnus sibirica Fisch. Ex Turcz. root extraction was subjected to ethyl acetate solvent fractionation using a seperatory funnel. A solvent fraction of the corresponding extract was filtered by filter paper (Hyundai Micro, Seoul, Korea) having a size of 297 mm×420 mm, and then concentrated by using a vacuum concentrator. Finally, through lyophilization, 74.39 g (yield 37.1%, ASRF-III) was secured in the form of a brown powder. 10 g of 17.2 g of powdered powder obtained through ASRF-I, a supercritical root extract of 60% ethanol extract of Alnus sibirica Fisch. Ex Turcz., was subjected to ethyl acetate solvent fractionation using seperatory funnel. A solvent fraction of the corresponding extract was filtered by filter paper (Hyundai Micro, Seoul, Korea) having a size of 297 mm×420 mm, concentrated by a vacuum concentrator, and freeze-dried to obtain 1.72 g (yield 17.2%, ASRF-II) in the form of a brown powder.

### 1.4. Separation Purification of MPLC

10 g of 74.39 g of a 60% ethanol extract ethyl acetate solvent fraction of root of Alnus sibirica Fisch. Ex Turcz. was dissolved in methyl alcohol at a concentration of 200 mg/mL, and then separation and purification was performed using MPLC of AI-580S HR-Premium (YAMAZEN, Osaka, Japan). As a first purification condition, a UNIVERSAL™ Premium Silica Gel 2L 3 cm×20 cm column from YAMAZEN and a inject column from Sillica gel L size were used. As a mobile phase solvent, Chloroform:Methanol:Water was mixed at a ratio of 70:30:4 and used, and as a mobile phase, Flow rate was analyzed at 20 mL/min. The Wavelength was analyzed at 254 nm and 280 nm, and the Inject volume was 5 mL and the total analysis time was 30 min. As a secondary purification condition for securing a single compound having high purity, a UNIVERSAL™ ODS-SM Gel L3 cm×16.5 cm column manufactured by YAMAZEN and a inject column manufactured by Silica gel L size were used. Methyl alcohol and Water were used as mobile phase solvents, and Flow rate was analyzed by 15 mL/min. The Wavelength was analyzed at 254 nm and 280 nm, and the inject volume was analyzed at 2 mL and the analysis time was analyzed at 40 min. The 2.42 g of brown powder material obtained by separation and purification was referred to as Compound 1.

In summary, in this example, the roots of Alnus sibirica Fisch. Ex Turcz were extracted using 60% alcohol, which is an edible alcohol, and a high content of extract was obtained with a solvent fraction of ethyl acetate, and then compound 1 was obtained through separation purification of MPLC. Molecular weight of the obtained compound 1 was analyzed by LC-MS/MS, and its structure was confirmed by NMR. analysis to confirm that it was oregonin. The present invention also relates to obtaining an extraction foil, which is a by-product remaining after supercritical extraction, performing 60% alcohol extraction to extract ASRF-I, and obtaining ASRF-II, which is an ethyl acetate solvent fraction of ASRF-I

### EXPERIMENTAL EXAMPLE

### 1, Material

### 1.1. Test Substance

Oregonin(ORE), a diarylheptanoid-based compound, which is an indicator and an effective material commonly detected in the entire area of a plant of the genus Alnus such as leaves, stems, roots, and woody parts of the plant of the genus Alnus, was separated and purified as described above and used as a test material.

### 1.2. Materials

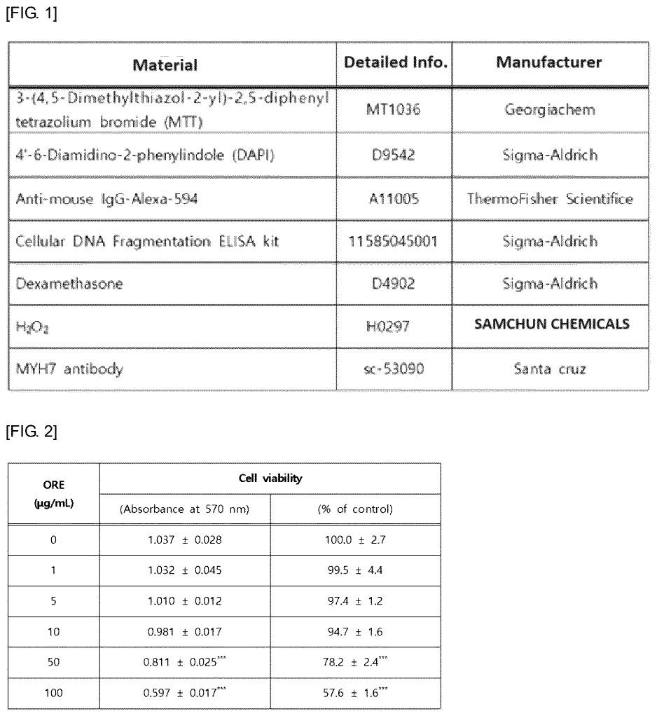

Experimental material information such as analysis kit used in the present invention is shown in the table of FIG. 1.

### 2. Method

### 2.1 Cell Culture

C2C12 cells, a myoblast derived from mouse skeletal muscle, were purchased from American Type Culture Collection (ATCC) and used. C2C12 cells were cultured in a 37° C.-wet CO2 incubator (5% CO2/95% air) using a cell culture medium in which 10% fetal bovine serum (FBS), 100 units/mL penicillin, and 100 μg/mL streptomycin were added to Dulbecco's Modified Eagle Medium (DMEM). When the cells were 80% full, the cell monolayer was washed with phosphate buffer saline (PBS, pH 7.4), trypsin-2.65 mM EDTA was added thereto, and the cells were detached and subcultured, and the medium was exchanged every 2 days. In order to induce differentiation into myotubes of C2C12 cells, the cells were cultured by exchange with a myocyte differentiation culture medium in which 2% horse serum (HS) was added to DMEM medium, and the myocyte differentiation culture medium was exchanged every 2 days.

### 2.2. Measurement of Cell Viability Under Normal Conditions

Cell viability of C2C12 cells was measured by MTT assay method (Denizot F and Lang R. J Immunological Method 89:271-277, 1986). culturing C2C12 cells for 48 hours by dividing the cells into 5×104 cells/well cells in 24-well plate, culturing the cells for 24 hours, and then exchanging cell culture fluid with a medium containing test materials of various concentrations (0-100 μg/mL)

It was. Thereafter, the cell culture medium was exchanged with a 1 mg/mL MTT (Amresco) solution, the cells were further cultured for 2 hours, and then formazan formed in living cells was eluted with isopropanol, and absorbance was measured at 570 nm to measure a cell viability.

### 2.3. Measurement of a Protective Effect on H2O2-Induced Myoblast Damage

C2C12 cells were seeded in 24-well plate at 5×104 cells/well and cultured for 24 hours. After culturing C2C12 cells for 24 hours, the cells were treated with 100 μM H2O2 to induce muscle cell damage, and the cells were cultured for 48 hours by treating 5 test substances with 100 μM H 2 O2 at various concentrations to investigate the effect of the test substance on protecting muscle cell damage. After the cells were cultured for 48 hours, MTT assay was performed in the same manner as described above to measure the cell viability.

### 2.4. Evaluation of Apoptosis of H2O2 Induced Myoblast

In order to evaluate the effect of the test substance on H2O2-induced myocyte apoptosis, C2C12 cells were seeded in 24-well plate at 5×104 cells/well and cultured for 24 hours. After culturing C2C12 cells for 24 hours, the cells were treated with 100 μM H2O2 to induce muscle cell damage, and the cells were cultured for 48 hours by treating 5 test substances with 100 μM H2O2 at various concentrations to investigate the effect of the test substance on protecting muscle cell damage. The degree of apoptosis of the myocytes was measured according to the method suggested by the manufacturer by using Cellular DNA Fragmentation ELISA kit (Sigma-Aldrich) which detects 5′-Bromo-2′-deoxy-uridine (BrdU)-labeled DNA.

### 2.5. Measurement of a Protective Effect on Dexamethasone Induced Myotube Cell Damage

C2C12 cells were seeded in 24-well plate at 5×104 cells/well and cultured for 24 hours. Thereafter, in order to induce differentiation into myotubes of C2C12 cells, the cell culture medium was exchanged with the myocyte differentiation culture medium to induce differentiation for 4 days. Thereafter, 5 μM dexamethasone was treated to induce muscle cell atrophy, and in order to investigate the effect of the test substance on protecting muscle cell damage, the cells were cultured for 24 hours by treating five test substances with 5 μM dexamethasone at various concentrations. After the cells were cultured for 24 hours, MTT assay was performed in the same manner as described above to measure the cell viability.

### 2.6. Measurement of a Protective Effect Against Dexamethasone Induced Myotube Cell Atrophy

C2C12 cells were cultured in 24-well plate containing cover glass for 24 hours to reach 5×104 cells/well. In order to induce differentiation into myotubes of C2C12 cells, the cell culture medium was exchanged with a myocyte differentiation culture medium to induce differentiation for 4 days. Thereafter, 5 μM dexamethasone was treated to induce muscle cell atrophy, and in order to investigate the effect of the test substance on protecting muscle cell damage, the cells were cultured for 24 hours by treating five test substances with 5 μM dexamethasone at various concentrations. The medium was removed, washed with PBS, and treated with 4% paraformaldehyde and 0.1% Triton X-100 to fix the cells. Blocking with 5% BSA/TBST and then primary antibody step (MYH7, santa cruz) was performed. Thereafter, the tissue was stained with secondary antibody (Anti-mouse IgG-Alexa-594, ThermoFisher Scientifice) and then counterstained with 4′-6-Diamidino-2-phenylindole (DAPI, Sigma-Aldrich) to investigate the expression of the protein using an optical microscope (Carl Zeiss).

### 2.7. Statistical Processing

All assay values were presented in mean±SEM. The collected results were analyzed using the GraphPad Prism 5.0 (GraphPad software, San Diego, Calif., USA) program. Student's t-test and one-way analysis variance (ANOVA) were used to compare the difference between the test substance-treated and the control. It was judged to be statistically significant only when p<0.05 or higher.

### 3. Results

### 3.1. Effect on Cell Viability of Myoblast

In order to examine the cytotoxicity of the test substance (ORE) in C2C12 cells, the test substance of ORE was treated with a cell culture medium at various concentrations (0, 1, 5, 10, 50, 100 μg/mL), cultured for 48 hours, and then subjected to MTT assay. Treatment with ORE significantly reduced cell viability from a concentration of 50 μg/mL compared to the control (0 μg/mL) (Table of FIG. 2). Based on the above results, a concentration that does not show cytotoxicity was set. That is, the highest treatment concentration of ORE was set to 10 μg/mL, and the following experiment was conducted.

### 3.2. Effect on H2O2-Induced Myoblast Damage

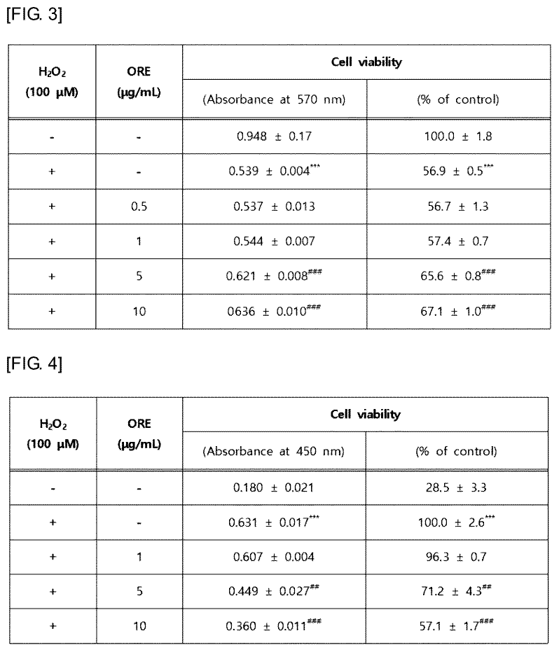

H2O2 (hydrogen peroxide) is a strong oxidant that induces oxidative stress in the in vitro system. In order to investigate the effect of the five test substances on the muscle cell damage caused by H2O2 induction, oxidative stress was induced by treating the cell culture fluid of C2C12 cells with 100 μM H2O2, and the cell viability of C2C12 cells was measured after 48 hours of incubation by treating the five test substances at various concentrations. As shown in the table of FIG. 3, the cell survival rate of the H2O2-treated group [H2O2 (+)/(−)] was significantly reduced compared to the control group [H2O2 (−)/(−)] not treated with H2O2. As shown in the table of FIG. 3, the cell viability was significantly increased at 5 μg/mL and 10 μg/mL treatment concentrations compared to the H2O2 treatment group [H2O2 (+)/(−)] treated with ORE (0.5, 1, 5, 10 μg/mL) (table of FIG. 3).

### 3.3. Effect on H2O2 Induced Myoblast Apoptosis

Oxidative stress is well known to cause cell death by causing DNA damage, and cell death by such oxidative stress as H2O2 occurs through an apoptosis process, which is a kind of programmed cell death. Therefore, in the present invention, segmented DNA was quantified using Cellular DNA Fragmentation ELISA kit in order to evaluate the effect of the five test substances on apoptosis due to oxidative stress. As shown in the table of FIG. 4, cell death was significantly increased in the H2O2-treated group [H2O2 (+)/(-)] compared to the control group [H2O2 (−)/(−)] not treated with H2O2.

Treatment with ORE significantly reduced apoptosis from 5μg/mL treatment concentration compared to H2O2 treatment group [H2O2 (+)/(−)], and reduced apoptosis by 42.9% at the highest treatment concentration of 10 μg/mL (table of FIG. 4).

### 3.4. Effect on Dexamethasone-Induced Myotube Cell Damage

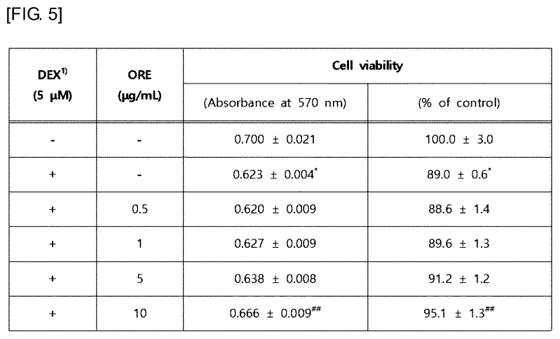

Dexamethasone is one of the representative glucocorticoid that causes the degradation of skeletal muscle in clinical misuse, and based on this, it is widely used to induce muscle cell atrophy in the in vitro system. In order to investigate the effect of the five test substances on the muscle cell damage caused by glucocorticoid, C2C12 cells were cultured with a muscle cell differentiation culture solution for 4 days to induce differentiation into myotubes, followed by treatment with 5 μM dexamethasone to induce muscle cell atrophy, and the viability of the myotubes was measured by treatment with the five test substances. As shown in the table of FIG. 5, the cell viability of the DEX-treated group [DEX (+)/(−)] was decreased by about 10% compared to the control group [DEX (−)/(−)] which was not treated with dexamethasone.

Treatment with ORE (0.5, 1, 5, 10 μg/mL) significantly increased cell viability at 10 μg/mL treatment concentration compared to DEX treatment group [DEX (+)/(−)] (table of FIG. 5).

In this test, in order to evaluate the alleviating effect in muscle loss of ORE commissioned by Dr. oregonin Co., Ltd. in the in vitro system, the effect of H2O2-induced muscle cell damage protection and the effect of dexamethasone-induced muscle cell atrophy protection were investigated.

In summary, the ORE significantly increased the cell viability of C2C12 cells (myoblasts), which was significantly reduced by H2O2 treatment, at a concentration at which the compound according to the present invention, ORE, did not affect the cell viability of C2C12 cells. In addition, ORE significantly decreased apoptosis of C2C12 cells (myoblasts), which was significantly increased by H2O2 treatment, and ORE significantly increased the survival rate of myocytes, which was decreased by dexamethasone treatment. Furthermore, the ORE protects damage to muscle cells induced by H2O2, protects muscle cell atrophy induced by dexamethasone, and may be confirmed as being capable of being developed into a material for treating or alleviating muscle loss (atrophy) in the future.

1. A pharmaceutical composition for preventing and treating muscle loss, comprising an oregonin compound derived from a plant of Alnus japonica as an active ingredient.

2. The pharmaceutical composition for preventing and treating muscle loss according to claim 1, wherein the oregonin compound derived from Alnus japonica plant is an extract of the supercritical extract cucurbitum of Alnus japonica plant.

3. The pharmaceutical composition for preventing and treating muscle loss according to claim 1, wherein the oregonin compound derived from the Alnus japonica plant is obtained by solvent fractionating the supercritical extract meal of the Alnus japonica.

4. The pharmaceutical composition for preventing and treating muscle loss of claim 3, wherein the Alnus japonica plant is a root of the Alnus japonica plant.

5.
A food composition for preventing and alleviating muscle loss, comprising an oregonin compound derived from a plant of Alnus japonica as an active ingredient.

6. The food composition for preventing and alleviating muscle loss of claim 5, wherein the oregonin compound derived from the Alnus japonica plant is an extract of the supercritical extract cucurbitum of the Alnus japonica plant.

7. The food composition for preventing and alleviating muscle loss according to claim 6, wherein the oregonin compound derived from the Alnus japonica plant is obtained by solvent fractionating the supercritical extract meal of the Alnus japonica.

8. The food composition for preventing and alleviating muscle loss of claim 5, wherein the Alnus japonica plant is a root of the Alnus japonica plant.

9. A feed composition for preventing and alleviating muscle loss, comprising an oregonin compound derived from a plant of Alnus japonica as an active ingredient.

10. The feed composition for preventing and alleviating muscle loss according to claim 9, wherein the oregonin compound derived from the Alnus japonica plant is an extract of the supercritical extract cucurbitus of the Alnus japonica plant.

11. The feed composition for preventing and alleviating muscle loss according to claim 10, wherein the oregonin compound derived from the Alnus japonica plant is obtained by solvent fractionating the supercritical extract meal of the Alnus japonica.

12. The feed composition for for preventing and alleviating muscle loss of claim 9, wherein the Alnus japonica plant is a root of the Alnus japonica plant.
